(19) United States
(12) Patent Application Publication
(10) Pub. No.: US 2026/0207251 A1
(43) Pub. Date: Jul. 23, 2026

(54) MYOTOMY CATHETER SYSTEM AND METHODS FOR A MYOTOMY CATHETER SYSTEM

(71) Applicant: The UNITED STATES OF AMERICA, as represented by the Secretary, Department of Health and Human Servic, Bethesda, MD (US)

(72) Inventors: Robert J. Lederman, Chevy Chase, MD (US); Dursun Korel Yildirim, Chevy Chase, MD (US)

(21) Appl. No.: 19/127,710

(22) PCT Filed: Nov. 8, 2023
(86) PCT No.: PCT/US2023/079114

§ 371 Date: May 6, 2025

Related U.S. Application Data
(60) Provisional application No. 63/383,012, filed on Nov. 9, 2022.

(51) Int. Cl. A61B 18/14; A61B 18/00

(52) U.S. Cl. CPC A61B 18/1492; A61B 2018/00279; A61B 2018/00351; A61B 2018/00577; A61B 2018/00601

(57) ABSTRACT

A myotomy catheter for performing longitudinal myocardial incisions is provided. In one example, a catheter-based heart incision apparatus comprises an anchor stabilization and orientation catheter system with at least one anchor element and an incision catheter including a lacerator configured to move along an incision trajectory oriented by the at least one anchor element.

### CROSS-REFERENCE TO RELATED APPLICATIONS

The present application is a U.S. National Phase of International Application No. PCT/US2023/079114, entitled “MYOTOMY CATHETER SYSTEM AND METHODS FOR A MYOTOMY CATHETER SYSTEM,” and filed on Nov. 8, 2023. International Application No. PCT/US2023/079114 claims priority to U.S. Provisional Patent Application No. 63/383,012, entitled “MYOTOMY CATHETER SYSTEM AND METHODS FOR A MYOTOMY CATHETER SYSTEM,” and filed Nov. 9, 2022. The entire contents of each of the above-listed applications are hereby incorporated by reference for all purposes.

### FIELD

The present description relates generally to a catheter system for cardiac procedures.

### BACKGROUND/SUMMARY

Some new intramyocardial structural heart procedures use catheter-based cardiac devices, which may be navigated into a targeted position within the heart muscle via a freely steered guidewire. As one example, septal scoring along the midline endocardium (e.g., SESAME) is a transcatheter myotomy procedure that may be used to relieve or prevent a left ventricular outflow tract (LVOT) obstruction by splaying the circumferential myofibers of the septal myocardium with a flying-V laceration surface formed by an ensnared guidewire tip previously navigated through the interventricular septum. Various imaging techniques may be used to provide navigation feedback during guidewire and/or cardiac device steering, such as x-ray fluoroscopy and echocardiography. However, x-ray fluoroscopy does not enable soft tissue visualization, causing the guidewire and/or cardiac device to appear free-floating in space. As another example, echocardiography is hindered by off-axis imaging planes, which may cause the guidewire and/or cardiac device to appear deeper or more shallow than it is actually positioned, or is impeded by other cardiac structures or implants or devices that interfere with imaging. As a result, it may be difficult to determine a radial position of the guidewire and/or cardiac device between endocardial and epicardial surfaces of the myocardium when performing intramyocardial device navigation using traditional imaging modalities. A radial depth of the cardiac device placement may affect procedure outcomes. For example, in the case of SESAME, the depth of the SESAME myotomy may be difficult to control. Excessively deep myotomy can cause ventricular septal defect or even free-wall perforation and death. Excessively shallow myotomy can fail to prevent or relieve LVOT obstruction, and can also fibrose and thereby reverse the myotomy.

In one example, the issues described above may be addressed by a catheter-based heart incision apparatus comprising an anchor stabilization and orientation catheter system with one or more mural and/or endocameral anchor elements and an incision catheter including a lacerator configured to move along an incision trajectory oriented by the one or more mural and/or endocameral anchor elements. In this way, the anchor element(s) may act to stabilize the incision catheter and allow the incision catheter to be positioned in a target location, without requiring the difficult image guidance and high operator skill that the conventional SESAME procedure may necessitate.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 depicts a first example myotomy catheter system according to a first embodiment of the disclosure, in a first configuration;

FIGS. 2 and 3 show the catheter system of FIG. 1 in a second configuration;

FIG. 4 is a side view of the catheter system of FIGS. 1-3 in the second configuration, including a plurality of cutting planes;

FIGS. 5-10 show cross-sectional views of the catheter system of FIG. 4 taken across each of the cutting planes;

FIG. 11 schematically shows the catheter system of FIGS. 1-10 inserted into a heart;

FIGS. 12A, 12B, and 12C depict a myotomy catheter system according to a second embodiment of the disclosure;

FIGS. 13A-13E depict a myotomy catheter system according to a third embodiment of the disclosure;

FIGS. 14-15 show a catheter system inserted into a heart;

FIG. 16 schematically shows positioning of the anchor ports and associated guidewires and catheter system disclosed herein, relative to myocardium;

FIG. 17 schematically shows positioning of a lacerator of the catheter systems disclosed herein, relative to myocardium;

FIGS. 18A-18D depict a second example of the myotomy catheter system according to the first embodiment of the disclosure;

FIGS. 19A-19D depict a third example of the myotomy catheter system according to the first embodiment;

FIG. 20 schematically shows the catheter system of the FIGS. 18A-18D inserted into a heart;

FIGS. 21A-21D depict a myotomy catheter system according to a fourth embodiment of the disclosure; and

FIGS. 22A and 22B show the catheter system of FIGS. 18A-18D overlaid on computed tomography images of a patient.

### DETAILED DESCRIPTION

The following description relates to a controlled-depth myotomy catheter system configured to perform a longitudinal endomyocardial myotomy, also referred to as septal scoring along the midline endocardium (SESAME), with precision and reproducibility, while demanding less operator skill than previous SESAME procedures, less reliance on image guidance, and lowered procedure times. SESAME is conventionally accomplished by navigating a 0.014″ guidewire under x-ray fluoroscopy and ultrasound guidance via a retrograde transaortic guiding catheter through the interventricular septum. A straight rigid engagement guidewire engages the basal septum either mechanically or electrosurgically to allow a 0.014″ microcatheter to enter the basal septum. The 0.014″ engagement guidewire is replaced with a 0.014″ curved-tip guidewire, which is navigated through the septal myocardium towards the apex until it exits the myocardium and reenters the left ventricular chamber. This septal intramyocardial guidewire trajectory defines the SESAME laceration thereafter performed. The trajectory may be confirmed by ultrasound. The intracameral guidewire tip is ensnared to allow positioning of an inner-curvature-denuded laceration surface resembling the “flying-V” used for other procedures such as laceration of the anterior mitral leaflet to prevent outflow obstruction (LAMPOON), except modified for SESAME. The “flying-V-like” configuration includes insulating microcatheters on both free limbs of the lacerating guidewire. Once positioned, the laceration surface is electrified under traction to accomplish SESAME. However, SESAME has important limitations that may impact safety and adoption. SESAME requires considerable operator experience and skill. Further, SESAME currently requires general anesthesia and advanced imaging including combinations of transthoracic, transesophageal, and intracardiac ultrasound, even within the left ventricular chamber, as well as biplane fluoroscopy. Ultrasound imaging windows across the chest wall, from the esophagus, and even within heart chambers are usually insufficient to give high confidence in the guidewire position along its whole trajectory. As explained previously, the uncertain depth of endomyocardial scoring via conventional SESAME may result in too shallow a laceration (resulting in therapeutic failure) or too deep a laceration (resulting in ventricular septal defect or free wall rupture).

The myotomy catheter system described herein (which may be referred to herein as a catheter-based heart incision apparatus) may address these issues by providing a controlled-depth laceration. The myotomy catheter system may be referred to as a transcatheter articulated heart incision instrument (TAHINI) catheter system. The TAHINI catheter system includes an incision catheter and an anchor stabilization and orientation catheter system configured to stabilize and properly orient the incision catheter. The anchor stabilization and orientation catheter system includes one or more anchors that are configured to be positioned in the heart to guide the incision catheter to perform SESAME. The anchor(s) may include mural anchors that enter or traverse the wall of the heart (e.g., the septum) and/or endocameral stabilizing hoops (that stabilize the catheter within a heart chamber, such as the left ventricle). The incision catheter may include an articulated cutting tool (e.g., a lacerator/blade that can be insulated from other metal components and electrified to perform electrosurgery) that, when deployed, is angled relative to the septal myocardium (e.g., tangential) such that traction-withdrawal along an anchored guidewire causes the cutting tool to embed deeply into the septal myocardium.

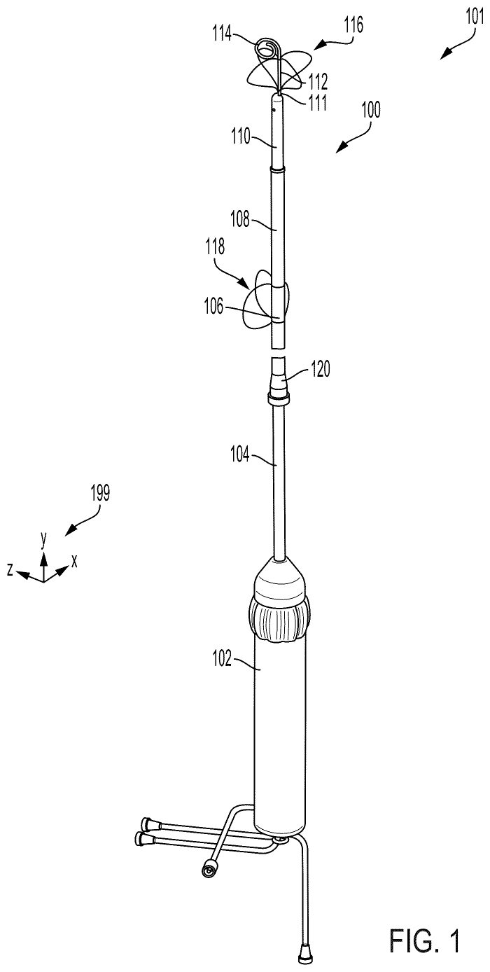

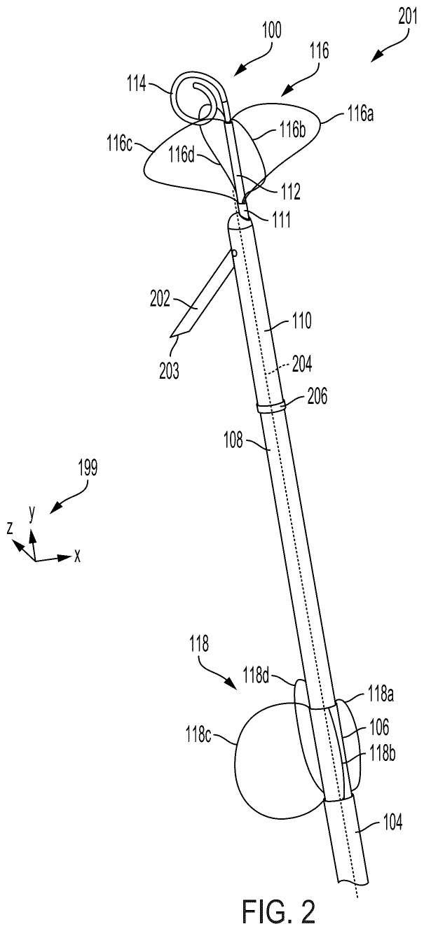

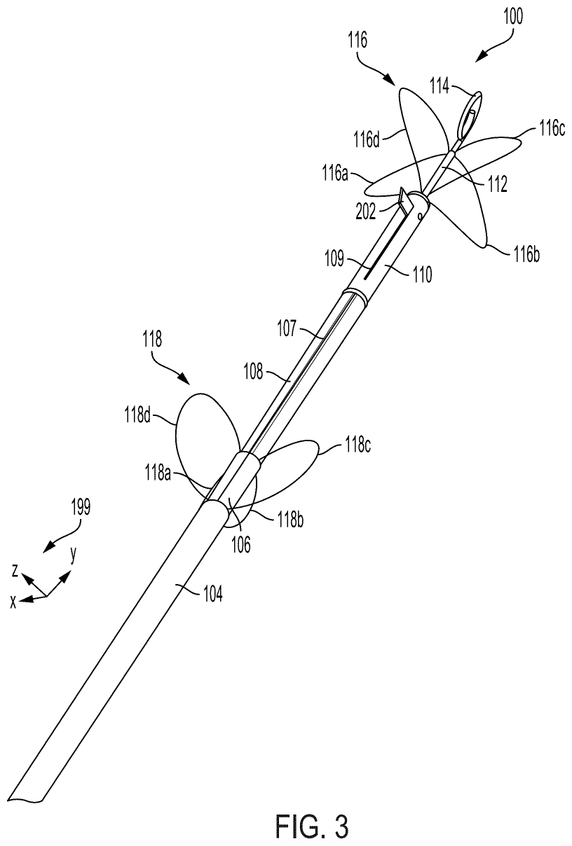

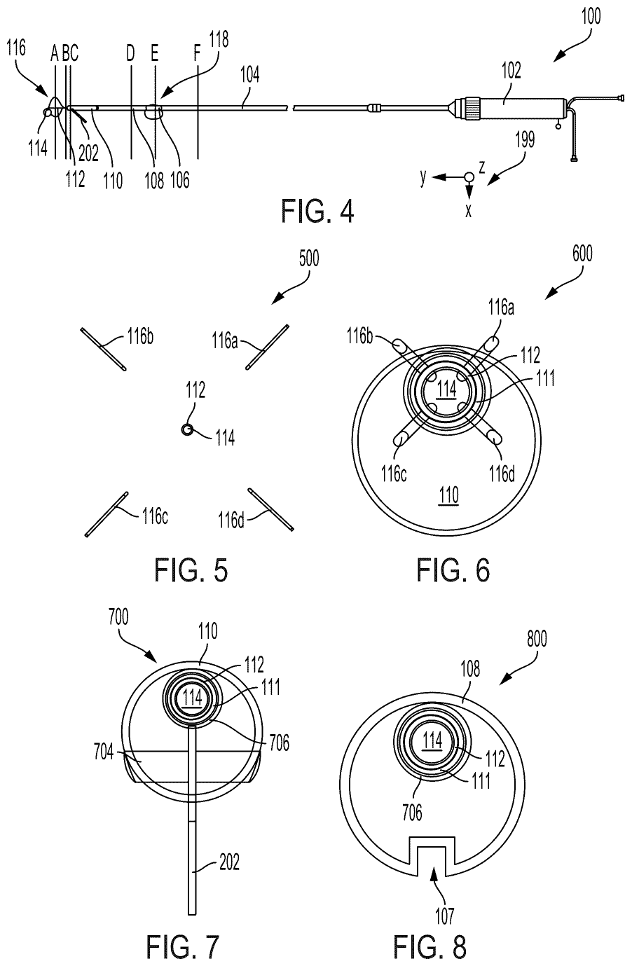

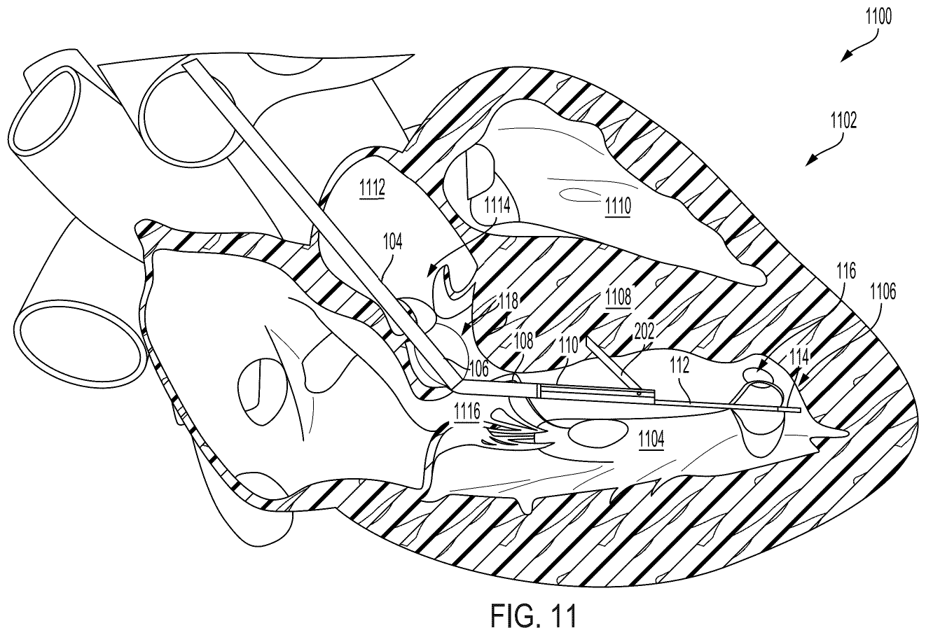

FIG. 1 shows a first example of a first embodiment of a TAHINI catheter system in a first configuration with a lacerator in a non-deployed (e.g., retracted) position. The TAHINI catheter system of the first embodiment includes a dedicated left ventricular cavitary anchor stabilization system that includes two sets of endocameral stabilizing hoop elements that may be deployed to position the incision catheter with regard to the target myocardium and to impart rotational stability. FIGS. 2 and 3 show magnified views of the distal end of the TAHINI catheter system of FIG. 1 in a second configuration with the lacerator deployed, while FIG. 4 shows a side view of the TAHINI catheter system of FIG. 1. FIGS. 5-10 show cross-sectional views of the TAHINI catheter system taken across cutting planes of FIG. 4. The TAHINI catheter system of FIGS. 1-10 may be used to carry out a SESAME procedure by inserting the TAHINI catheter system in a heart of a patient, as shown in FIG. 11.

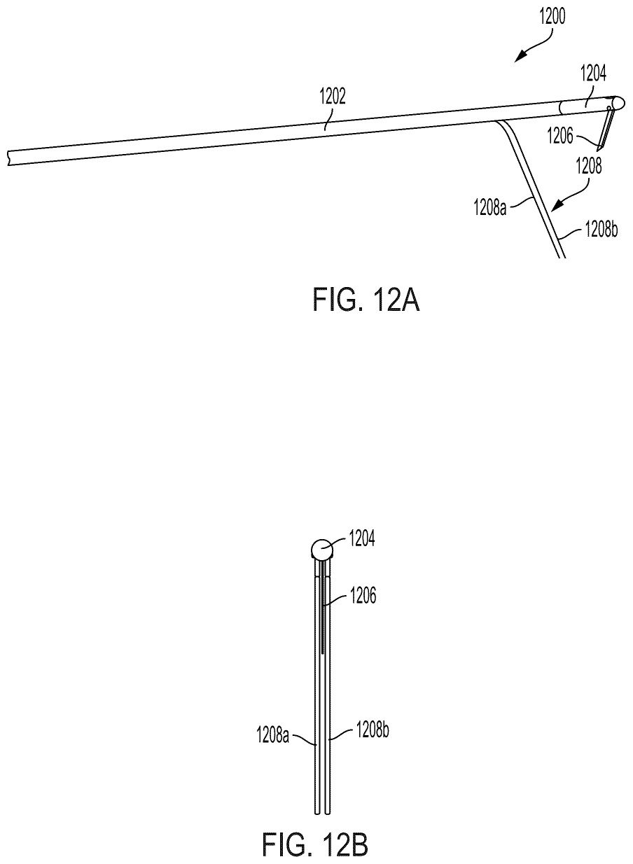

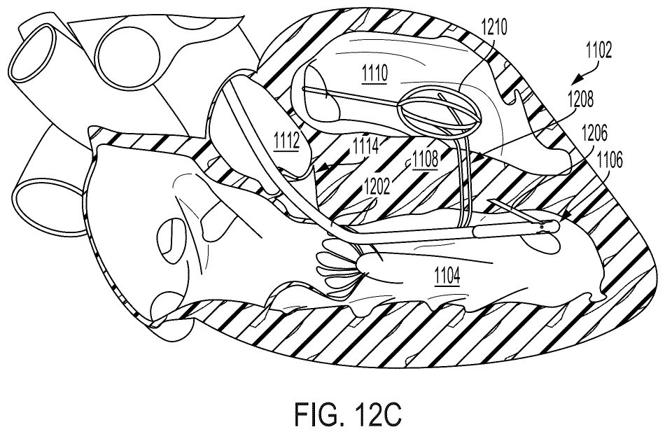

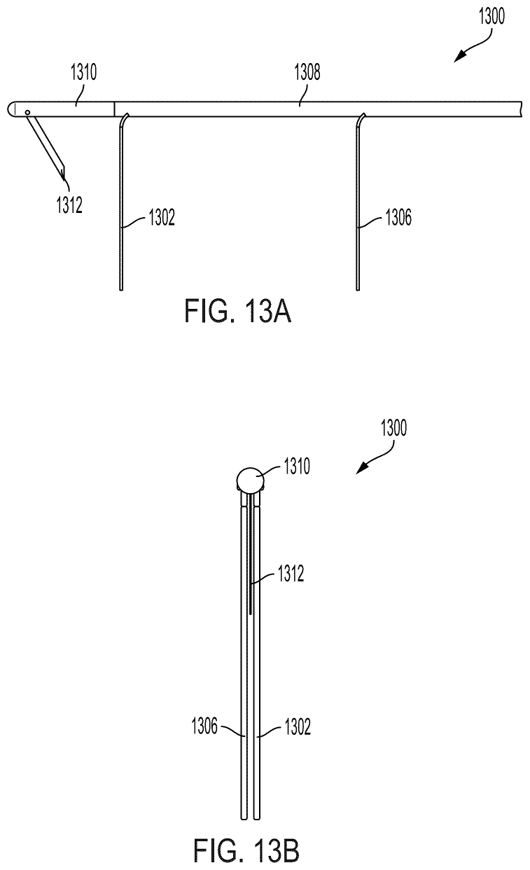

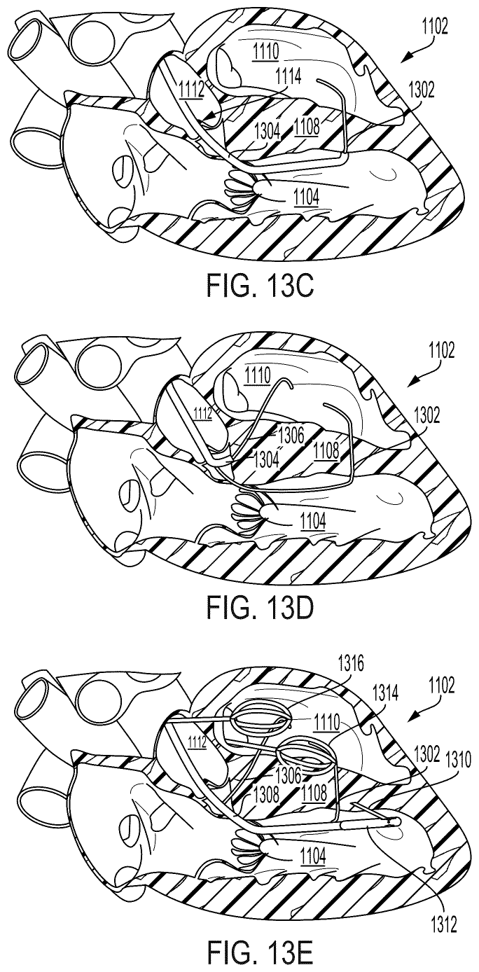

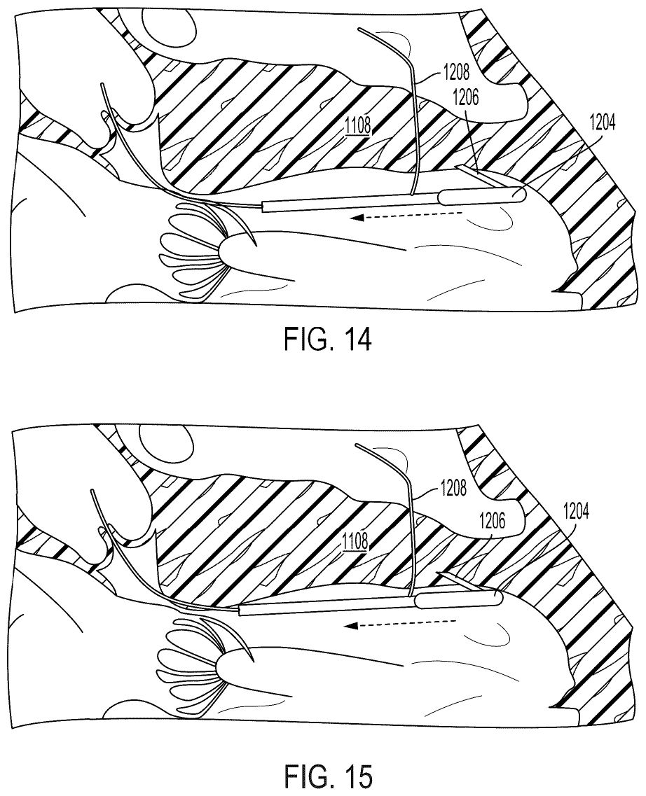

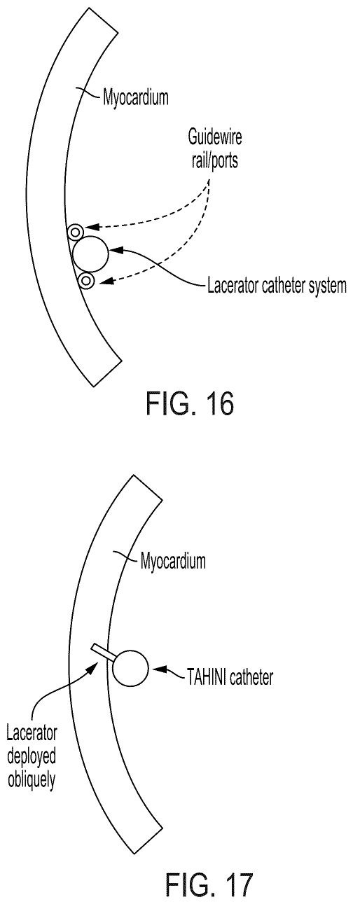

FIGS. 12A and 12B show a second embodiment of a TAHINI catheter system with a lacerator in a deployed position. The TAHINI catheter system of FIGS. 12A and 12B includes two anchor ports to accommodate two parallel transmyocardial/mural anchors placed radially into the right ventricle (e.g., right ventricular space) that may be deployed to position the incision catheter with regard to the target myocardium and to impart rotational stability. FIG. 12C illustrates the TAHINI catheter system of FIGS. 12A and 12B inserted into a heart of a patient to perform a SESAME procedure. FIGS. 13A-13E show a third embodiment of a TAHINI catheter system with a lacerator in a deployed position. The TAHINI catheter system of FIGS. 13A-13E includes one or more anchor ports to accommodate distal and optional proximal transmyocardial/mural anchors placed radially into the right ventricle (e.g., right ventricular space) that may be deployed to position the incision catheter with regard to the target myocardium and to impart rotational stability. FIGS. 13C-13E schematically illustrate the TAHINI catheter system of FIGS. 13A-13B inserted into a heart of a patient to perform a SESAME procedure. FIGS. 14-15 illustrate a TAHINI catheter system, such as the catheter system of FIG. 12A or 13A inserted into a heart of a patient to perform a SESAME procedure. FIGS. 16 and 17 schematically show positioning of aspects of the TAHINI catheter systems disclosed herein relative to myocardium.

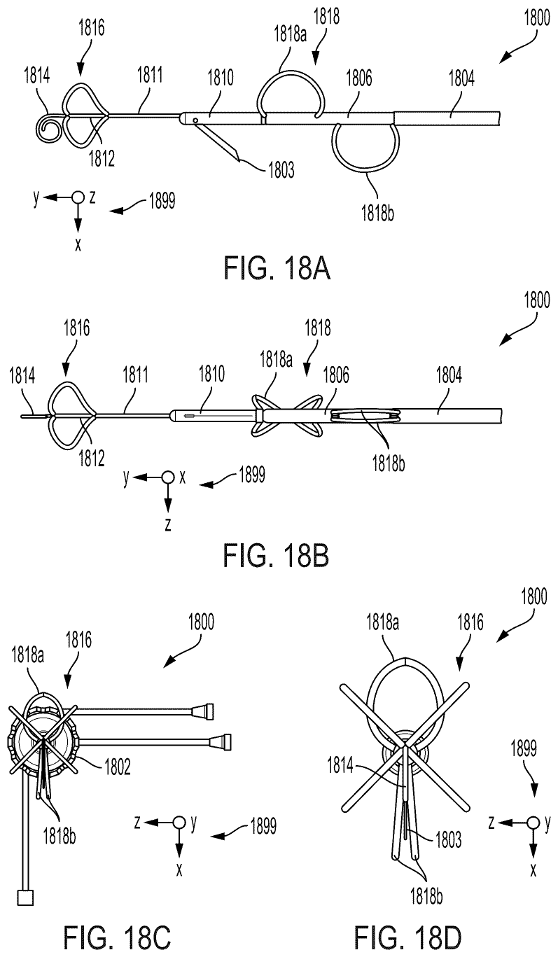

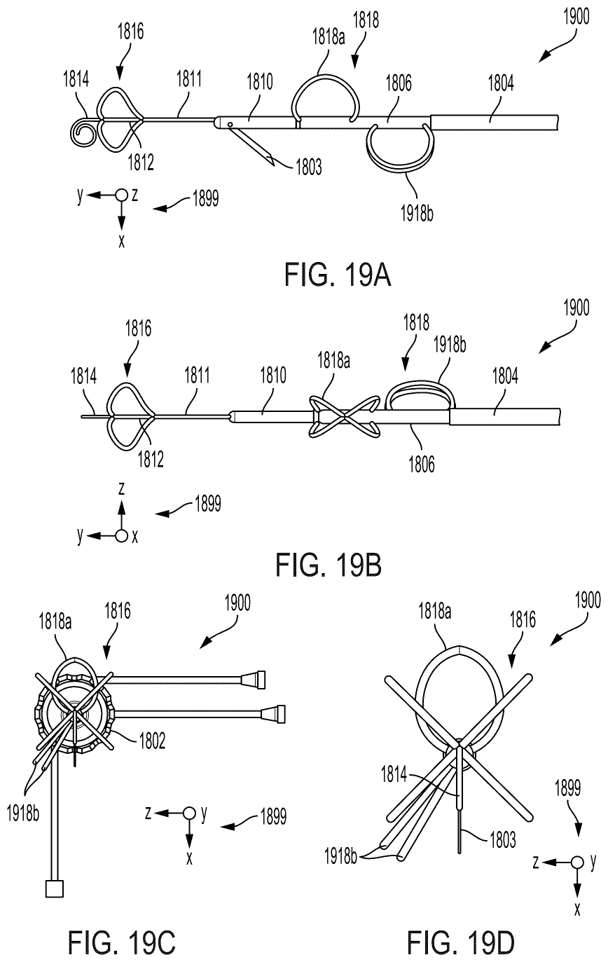

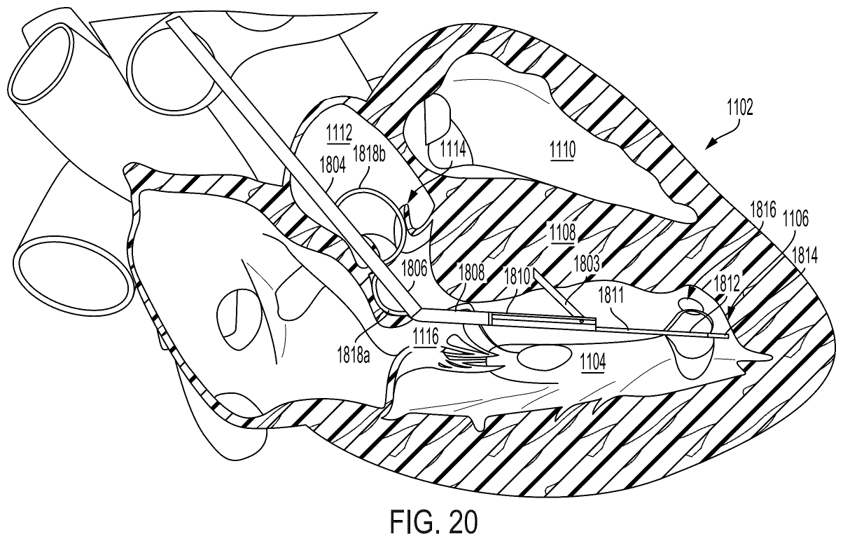

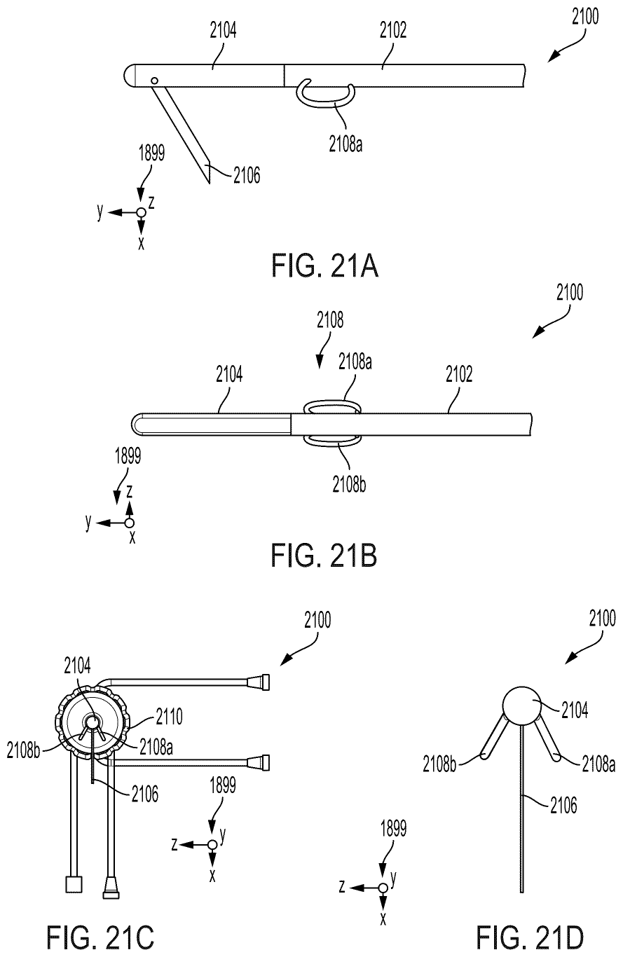

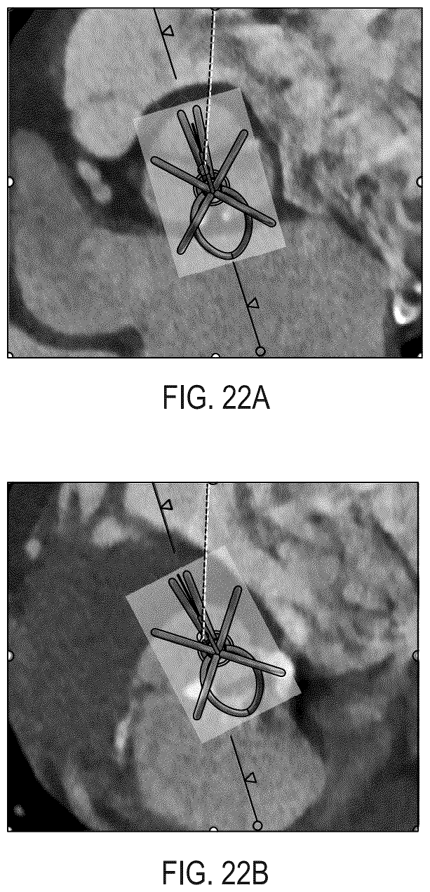

FIGS. 18A-18D show a second example of the TAHINI catheter system of the first embodiment with endocameral stabilizing hoop elements that may be deployed to position the incision catheter with regard to the target myocardium and to impart rotational stability. The catheter system of the FIGS. 18A-18D includes apical stabilizing hoop elements, similar to the first example, and further includes proximal stabilizing hoop elements that are offset longitudinally from each other in order to straddle the aortic valve. In some examples, aspects of the catheter system may be keyed to impart rotational stability and position the stabilizing hoop elements with respect to the lacerator, as shown by a second example of the catheter system of the first embodiment in FIGS. 19A-19D. FIG. 20 schematically illustrates the TAHINI catheter system of FIGS. 18A-18E inserted into a heart of a patient to perform a SESAME procedure while FIGS. 22A and 22B show the catheter system of FIGS. 18A-18D overlaid on images of a heart of a patient. FIGS. 21A-21D show a fourth embodiment of a TAHINI catheter system with deployable anchors that may be deployed to position the incision catheter with regard to the target myocardium and to impart rotational stability. FIGS. 1-10 and 12A, 12B, 13A, 13B, 18A-18D, 19A-19D, and 21A-21D are drawn to scale, but other relative dimensions could be used, if desired.

Turning now to the figures, FIG. 1 depicts a first example of a myotomy catheter system 100 (also referred to as a TAHINI catheter system) according to a first embodiment of the disclosure, in a first configuration 101. FIG. 1 (as well as FIGS. 2-4) includes a Cartesian coordinate system 199 to orient each view of the catheter system 100 provided herein. In the example shown in FIG. 1, the y-axis extends parallel to gravity with the positive y direction pointing in the direction of the arrow, away from ground. However, it is to be appreciated that the catheter system 100 may be held or used in any orientation without departing from the scope of this disclosure. Further, the term distal end may refer to a first end of the catheter system 100 configured to be positioned within a heart of a patient and the term proximal end may refer to a second end of the catheter system 100 configured to remain external to the patient and including a handle, as explained below.

The catheter system 100 includes a handle 102 and outer catheter shaft 104 to position (advance, withdraw, rotate) aspects of the catheter system 100, to control an incision catheter (extend or retract, including variably and interactively), and to electrify an electrosurgical traversal and laceration surface of a cutting tool of the incision catheter, and optionally to indicate the rotational position based radiographic attenuating markers and/or to inject non-ionic flush or angiographic contrast. The outer catheter shaft 104 houses a plurality of coaxial tubes including a first inner tube 106 and a second inner tube 108. The second inner tube 108 may be positioned inside and coaxial with the first inner tube 106. The second inner tube 108 houses an incision catheter 110. As will be explained in more detail below, the incision catheter 110 includes a deployable lacerator that may be embedded in myocardium, such as in the septum to perform a SESAME procedure. In the first configuration 101 shown in FIG. 1, the lacerator is not deployed and is instead housed within the incision catheter 110. Extending outward from the incision catheter 110 is a third inner tube 111, which houses a coaxial, fourth inner tube 112. The fourth inner tube 112 may act as a guidewire lumen to accommodate a guidewire 114. Also shown in FIG. 1 is a hub 120, which may be used to anchor the outer catheter shaft 104 to an exterior of a patient, for example, such that the components of the catheter system 100 positioned distal to the hub 120 are configured to be positioned in the patient during a cardiac procedure (e.g., SESAME) while the remaining portion of the outer catheter shaft 104 and handle 102 are configured to remain external to the patient. It is to be appreciated that outer catheter shaft 104 may have a suitable length that is longer than shown in FIG. 1 and that a middle portion of the outer catheter shaft 104 has been removed herein for visual clarity (shown by the double lines in FIG. 1).

The catheter system 100 includes two sets of endocameral stabilizing hoop elements, a first set of hoop elements 116 positioned at the distal end of the catheter system 100 and a second set of hoop elements 118 positioned proximal to the incision catheter 110. The first set of hoop elements 116 may be coupled to the fourth inner tube 112 and may expand and contract based on a position of the third inner tube 111. Thus, the fourth inner tube 112 and the third inner tube 111 form a coaxial two-tube system to expand/contract the first set of hoop elements 116. In the example shown herein, the first set of hoop elements 116 includes four hoop elements, though it is to be appreciated that more or fewer hoop elements could be included without departing from the scope of this disclosure. Each hoop element of the first set of hoop elements 116 may be constructed of round or flat metallic elements that can be expanded/contracted under coaxial catheter control to contact the left ventricular apex and thereby impart longitudinal and rotational stability. The first set of hoop elements 116 may therefore comprise apical hoops, and may be pre-shaped as petal-shaped elements, and the degree of their extension is controlled by the telescoping third inner tube 111.

The second set of hoop elements 118 may be coupled to the first inner tube 106 and may expand and contract based on a position of the outer catheter shaft 104. Thus, the first inner tube 106 and the outer catheter shaft 104 form an outer coaxial two-catheter system to expand/contract the second set of hoop elements 118. In the example shown herein, the second set of hoop elements 118 includes four hoop elements, though it is to be appreciated that more or fewer hoop elements could be included without departing from the scope of this disclosure. Each hoop element of the second set of hoop elements 118 may be constructed of round or flat metallic elements that can be expanded/contracted under coaxial catheter control to contact the left ventricular outflow tract (LVOT) and right coronary cusp (RCC) of the aortic valve, and thereby impart longitudinal and rotational stability. The second set of hoop elements 118 may therefore comprise LVOT/RCC hoops, and may be pre-shaped as petal-shaped elements, and the degree of their extension is controlled by the telescoping outer catheter shaft 104. Further, the LVOT/RCC petals (e.g., the second set of hoop elements 118) may be asymmetric to position the catheter system 100 eccentrically along the LVOT/RCC to position the incision catheter against the intended target tissue, such as septum. For example, the second set of hoop elements 118 may include two larger hoop elements and two smaller hoop elements, as described in more detail below with respect to FIGS. 2 and 3.

FIGS. 2 and 3 show magnified views of the distal end of the catheter system 100 in a second configuration 201, where a lacerator 202 of the incision catheter 110 is deployed. FIGS. 2 and 3 will be described collectively. It is to be appreciated that FIG. 3 shows the catheter system 100 rotated around its central longitudinal axis 204 shown in FIG. 2 (e.g., parallel to the y axis of coordinate system 199) relative to the view of FIG. 2. The lacerator 202 may be connected to an actuating element, such as opposed tensioning wires, a worm gear, or a push/pull-rod, that extends from the incision catheter to the handle 102, such that a user may deploy or retract the lacerator 202 as well as move the lacerator 202 along its incision trajectory via actuation of the actuating element. The incision catheter 110 may include a lacerator opening 109 (shown in FIG. 3) that extends longitudinally along the incision catheter 110, and the lacerator 202 may sit within the lacerator opening 109 when the lacerator 202 is retracted. The lacerator 202 may have a suitable length, such as 20 mm, and a suitable width, such as 4 mm, depending on the target radial depth of incision in the myocardium. The lacerator 202 may include one or more cutting edges, such as edge 203, which may be thinner/sharper than other edges of the lacerator 202.

When deployed, the lacerator 202 may extend at an oblique angle relative to the incision catheter 110. The incision catheter 110 extends parallel to (and is collinear with) the central longitudinal axis 204. The lacerator 202 may extend to a maximum angle relative to the incision catheter 110/central longitudinal axis 204 that is less than 90 degrees. In some examples, the lacerator 202 may extend in a range of angles, such as within a range of 0 to 60 degrees. When the lacerator 202 is at an angle of 0 degrees, the lacerator 202 may be fully retracted and housed within the incision catheter 110. In the example shown in FIG. 2, the lacerator 202 is extended at an angle of 45 degrees. The lacerator deflection can be adjusted between the range of angles (e.g., 0 to 60 degrees) to enable a target maximum radial depth of incision in the myocardium, such as maximum radial depth of 16 mm. Shorter lacerators may be suitable for indications demanding more shallow lacerations, while longer lacerators may be suitable for indications demanding deeper lacerations. In some examples, the lacerator 202 may be supplied electricity (e.g., via the actuating element) to conduct electrosurgery. In such examples, parts of the lacerating surfaces may be insulated to concentrate energy deposition on the cutting edge (e.g., edge 203) of the lacerator and to reduce alternative current paths beyond the intended laceration surface. For example, an insulating material may be present over a portion of the lacerator to form an exposed monopole of the lacerator that is configured to effect electrosurgical laceration. The lacerator 202 may be comprised of suitable material(s), such as electrically-conductive metal (e.g., stainless steel, copper, etc.) and/or may include one or more electrodes to impart ablative electrosurgical energy. In some examples, the catheter system 100 may include a feedback electrode that may be used to provide feedback regarding a radial position (also termed radial depth) of the lacerator within the myocardium. For example, the feedback electrode may be positioned on the lacerator (e.g., a distal end of the lacerator) and be configured to obtain electrical signals of the heart that may be analyzed to determine a relative depth of the lacerator in the myocardium.

As mentioned previously, the catheter system 100 includes two sets of endocameral stabilizing hoop elements. FIGS. 2 and 3 show the first set of hoop elements 116 and the second set of hoop elements 118 in more detail. The first set of hoop elements 116 includes four apical hoop elements, a first hoop element 116a, a second hoop element 116b, a third hoop element 116c, and a fourth hoop element 116d. The four apical hoop elements may be distributed equidistantly around the fourth inner tube 112 and positioned at the same longitudinal location on the fourth inner tube 112 (e.g., each hoop element of the first set of hoop elements 116 may terminate/couple to the fourth inner tube 112 at the same locations along the y axis/longitudinal axis 204). Each hoop element of the first set of hoop elements 116 may be made of the same material and may have the same shape and size. As appreciated from FIG. 2, the third inner tube 111 and the fourth inner tube 112 may extend from the incision catheter 110 eccentrically (e.g., not from the centremost point of the top of the incision catheter 110), such that the third inner tube 111 and the fourth inner tube 112 have a central longitudinal axis that is parallel to but not collinear with the central longitudinal axis 204, which may allow space in the incision catheter 110 to accommodate the lacerator 202.

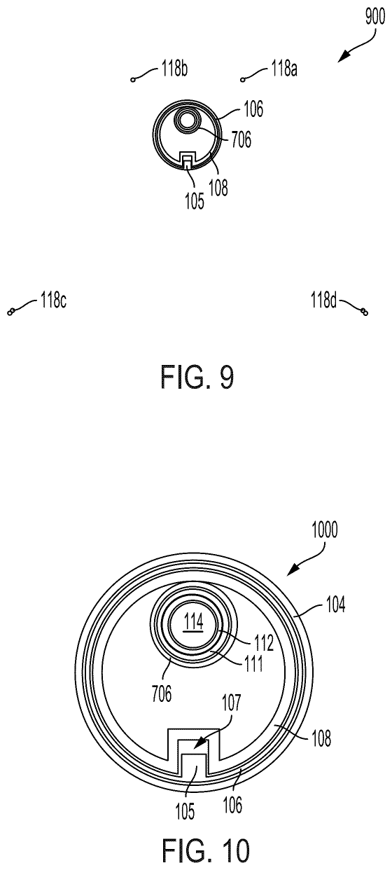

The second set of hoop elements 118 includes four hoop elements, a first hoop element 118a, a second hoop element 118b, a third hoop element 118c, and a fourth hoop element 118d. The four hoop elements of the second set of hoop elements 118 may be distributed equidistantly around the first inner tube 106 and positioned at the same longitudinal location on the first inner tube 106 (e.g., each hoop element of the second set of hoop elements 118 may terminate/couple to the first inner tube 106 at the same locations along the y axis/longitudinal axis 204). Each hoop element of the second set of hoop elements 118 may be made of the same material. The hoop elements of the second set of hoop elements 118 may have different sizes and/or shapes. For example, the first hoop element 118a and the second hoop element 118b may be sized and shaped similarly to each other, but may be smaller than the third hoop element 118c and the fourth hoop element 118d. For example, the third hoop element 118c and the fourth hoop element 118d may extend outward from the first inner tube 106 to a larger degree than the first hoop element 118a and the second hoop element 118b, forming partial circles of greater diameter than the first hoop element 118a and the second hoop element 118b. As appreciated from FIG. 3, the third hoop element 118c and the fourth hoop element 118d may be coupled to and extend outward from a first radial portion of the first inner tube 106, which may define a first, lacerator side of the catheter system 100, while the first hoop element 118a and the second hoop element 118b may be coupled to and extend outward from a second radial portion of the first inner tube 106, which may define a second, opposite side of the catheter system 100. The lacerator 202 may be positioned on and extend from the first side. The incision catheter 110 may be maintained in the rotational position shown in FIG. 3 relative to the first inner tube 106 and the second inner tube 108 (such that the lacerator opening 109 and hence the lacerator 202 are positioned along a line that extends between the third hoop element 118c and the fourth hoop element 118d) via keying elements that include a keying notch 107 on the second inner tube 108 configured to accommodate a keying protrusion 105 on the inner surface of the first inner tube 106 (not visible in FIGS. 2 or 3, but shown in FIGS. 9 and 10 and explained in more detail below). Similar keying elements may be included to maintain the rotational position of other tubes, such as the incision catheter relative to the second inner tube 108 (e.g., the incision catheter 110 may include a notch configured to accommodate a protrusion on the second inner tube 108), the first inner tube 106 relative to the outer catheter shaft 104 (e.g., the first inner tube 106 may include a keying notch that is configured accommodate a keying protrusion of the outer catheter shaft 104), the fourth inner tube 112 relative to the third inner tube 111, etc. Further, in some examples, a stopper 206 may be positioned on the apical and/or LVOT stabilization element (e.g., on the third inner tube 111 and/or on the second inner tube 108, as shown) to limit the longitudinal movement of the incision catheter 110. Additionally, in some examples, one or more of the inner tubes and/or the incision catheter 110 may include a dedicated flush port through a sidearm or the like.

FIG. 4 shows a side view of the catheter system 100 with cutting planes for various cross-sectional views (shown in FIGS. 5-10) depicted by the letters A-F. FIG. 5 illustrates a first cross-sectional view 500 taken across line A of FIG. 4. The first cross-sectional view 500 shows the cross-section of the distal end of the catheter system 100 and thus includes the fourth inner tube 112 housing the guidewire 114. Each hoop element of the first set of hoop elements 116 is shown, with the cutting plane cutting through the widest portion of each hoop element to show a distal edge formed by the cutting plane and the hoop element extending behind the distal edge. The first cross-sectional view 500 is a zoomed out view relative to other cross-sectional views shown in FIGS. 6-8 and 10, in order to visualize the hoop elements of the first set of hoop elements 116 along with the fourth inner tube 112 and guidewire 114.

FIG. 6 illustrates a second cross-sectional view 600 taken across line B of FIG. 4. The second cross-sectional view 600 shows a cross-section of the third inner tube 111 where the third inner tube 111 exits the distal end of the incision catheter 110. In the second cross-sectional view 600, the third inner tube 111, the fourth inner tube 112, and the guidewire 114 are shown in cross-section, along with each hoop element of the first set of hoop elements 116. The distal outer surface of the incision catheter 110 is shown, extending behind the cross-sectional views of the third inner tube 111, the fourth inner tube 112, the guidewire 114, and each hoop element of the first set of hoop elements 116. As explained previously, the third inner tube 111 (and hence fourth inner tube 112, the guidewire 114, and the first set of hoop elements 116) is positioned off-center relative to the incision catheter 110. Further, each hoop element of the first set of hoop elements 116 extends from the proximal end of the fourth inner tube 112 and between the fourth inner tube 112 and the third inner tube 111. The components shown in FIG. 6 may have suitable cross-sectional diameters that may be selected to allow the catheter system to be navigated into the left ventricular space, as will be explained in more detail below with respect to FIG. 11. For example, the guidewire 114 may be a 0.035-inch guidewire and thus may have a diameter of 0.89 mm. The fourth inner tube 112 may have a diameter of 1.17 mm where the fourth inner tube 112 extends from the third inner tube 111. The third inner tube may have a diameter of 1.40 mm. The incision catheter 110 may have a diameter of 4.00 mm. However, other dimensions may be used without departing from the scope of this disclosure.

FIG. 7 illustrates a third cross-sectional view 700 taken across line C of FIG. 4. The third cross-sectional view 700 shows a cross-section of the incision catheter 110 where the lacerator 202 is coupled to the incision catheter 110 when the lacerator 202 is deployed. FIG. 7 shows a pivot pin 704 for rotation of the lacerator 202 and a port lumen 706 for accommodating the distal stabilizer catheters that enables the lacerator 202 to be retracted/deployed and couples the lacerator 202 to internal aspects of the catheter system 100. In the example shown herein, the lacerator 202 is expanded/contracted via actuation of tensioner wires placed behind the lacerator, though other actuation elements may be used without departing from the scope of this disclosure. As explained above, the radial portion of the incision catheter 110 that includes the lacerator 202 may be referred to as the lacerator side of the catheter system 100, which in the view shown in FIG. 7 (and the other views shown in FIGS. 4-10) is the bottom side. The port lumen 706, as well as concentrically arranged third inner tube 111, fourth inner tube 112, and guidewire 114 are positioned on the opposite (e.g., top) side of the catheter system 100.

FIG. 8 illustrates a fourth cross-sectional view 800 taken across line D of FIG. 4. The fourth cross-sectional view 800 shows a cross-section of the second inner tube 108, with the port lumen 706, the third inner tube 111, the fourth inner tube 112, and the guidewire 114 housed concentrically therein. The line D, and hence the fourth cross-sectional view 800, is positioned proximally relative to the incision catheter 110 and hence the incision catheter 110 is not present in the illustrated section of the second inner tube 108. The second inner tube 108 includes the keying notch 107 on the lacerator side of the outer surface of the second inner tube 108. As shown, the keying notch 107 is a rectangular-shaped notch that extends a suitable distance (e.g., 10% of the diameter) into the second inner tube 108. However, the keying notch 107 could have another shape without departing from the scope of this disclosure, such as a circular shape.

FIG. 9 illustrates a fifth cross-sectional view 900 taken across line E of FIG. 4. The fifth cross-sectional view 900 shows a cross-section of the first inner tube 106 positioned around the second inner tube 108, with the port lumen 706, the third inner tube 111, the fourth inner tube 112, and the guidewire 114 positioned concentrically within the second inner tube 108 (only the port lumen 706 is numbered in FIG. 9 for visual clarity). The first inner tube 106 includes a keying protrusion 105 on the inner surface of the first inner tube 106, on the lacerator side. The keying protrusion 105 is shaped and sized to fit within the keying notch 107 of the second inner tube 108, in order to maintain the second inner tube 108 in radial position relative to the first inner tube 106. The fifth cross-sectional view 900 further shows a cross-section of the second set of hoop elements 118, illustrating the smaller size (and hence closer position relative to the first inner tube 106) of the first hoop element 118a and the second hoop element 118b compared to the third hoop element 118c and fourth hoop element 118d. In a non-limiting example, the third hoop element 118c and the fourth hoop element 118d may extend out from the first inner tube 106 to a maximum width of 6.6 mm while the first hoop element 118a and the second hoop element 118b may extend out from the first inner tube 106 to a maximum width of 0.8 mm. Similar to FIG. 5, the fifth cross-sectional view 900 is a zoomed out view relative to other cross-sectional views shown in FIGS. 6-8 and 10, in order to visualize the hoop elements of the second set of hoop elements 118 along with the first inner tube 106.

FIG. 10 illustrates a sixth cross-sectional view 1000 taken across line F of FIG. 4. The sixth cross-sectional view 1000 shows a cross-section of the outer catheter shaft 104 housing the first inner tube 106, the second inner tube 108, the port lumen 706, the third inner tube 111, the fourth inner tube 112, and the guidewire 114, all arranged concentrically within the outer catheter shaft 104. The second inner tube 108 may have a diameter of 4.38 mm. The first inner tube 106 may have a diameter of 4.40 mm. The outer catheter shaft 104 may have a diameter of 4.70 mm. However, other dimensions are possible without departing from the scope of this disclosure.

As appreciated by the cross-sectional views of FIGS. 5-10, the catheter system 100 is configured to maintain certain components in position relative to each other in order to stabilize the incision catheter 110 and allow the lacerator 202 to be accurately positioned for incising the myocardium. The keying elements described above (e.g., keying notch 107 and keying protrusion 105) may assist in maintaining the second inner tube 108 in rotational position relative to the first inner tube 106. The third inner tube 111 and the fourth inner tube 112 may be rotationally constrained by the opening in the incision catheter 110 through which the third inner tube 111 extends. The third inner tube 111 and the fourth inner tube 112 are at an off-centric location which is almost symmetrical to the keyed first inner tube 106 and second inner tube 108. The torque required to rotate the lacerator around the longitudinal axis may be limited by the keyed components and vice versa.

FIG. 11 shows a cross-sectional view 1100 of a heart 1102 with the catheter system 100 positioned therein. The heart 1102 includes a left ventricular space 1104 (also referred to as a chamber) having an apex 1106. An interventricular septum (IVS) 1108 is positioned between the left ventricular space 1104 and a right ventricular space 1110. The heart 1102 shown in FIG. 11 may have an LVOT obstruction whereby the LVOT (e.g., via a mitral valve 1116) is obstructed by thickening of the IVS 1108 (e.g., as a result of hypertrophic cardiomyopathy). To relieve the obstruction, the catheter system 100 may be positioned in the left ventricular space 1104 to enable the SESAME procedure to be performed, which may include embedding the lacerator 202 in the IVS 1108 and incising the IVS 1108 longitudinally with the lacerator 202, which causes the cut circumferential myofibers of the IVS 1108 to splay apart, reducing septal encroachment into the LVOT without removal of muscle tissue and thereby relieving the LVOT obstruction.

The SESAME procedure may be performed by guiding the catheter system 100 over a LV apical guidewire (e.g., guidewire 114) retrograde through the aorta 1112 and across the aortic valve 1114, and the distal end of the catheter system 100 may be positioned near the LV apex 1106. The apical hoop elements (e.g., the first set of hoop elements 116) may be extended by advancing the coaxial tubes (e.g., the fourth inner tube 112 and the third inner tube 111) controlling the first set of hoop elements through the handle 102. Then the LVOT hoop elements (e.g., the second set of hoop elements 118) may be positioned similarly in the LVOT. The position of the catheter system 100 may be confirmed by ultrasound and by angiography, for example. The target position for the catheter system 100 may include the second set of hoop elements 118 being positioned between the IVS 1108 and the aortic valve 1114, with the larger hoop elements (e.g., the third hoop element 118c and the fourth hoop element 118d) facing/in contact with the IVS 1108. The target position may further include the first set of hoop elements 116 contacting the myocardium at the apex 1106. This positioning may allow the lacerator 202, when deployed as shown in FIG. 11, to face the IVS 1108 at an oblique angle.

In some examples, before the catheter system 100 is guided into the left ventricular space 1104, the target position for the catheter system 100 may be determined based on pre-operative diagnostic images of the heart 1102, such as computed tomography images, which may allow visualization of the geometry of the LVOT, including intended transcatheter heart valve deployment and its impact on fixed and dynamic LVOT obstruction. From the diagnostic images, planning information for the SESAME procedure may be obtained/determined, such as the anchor site (e.g., the position at which the distal end of the catheter system 100 is placed), the distal and proximal laceration start-and end-positions, and the laceration depth. Further, in some examples, a specific catheter system may be selected based on the diagnostic images/LVOT geometry that can incise with the determined laceration depth (e.g., a catheter system having a certain lacerator length and/or maximum lacerator angle may be selected for performing the SESAME procedure). Further still, desired radiographic projection angles may be selected based on the diagnostic images/LVOT geometry.

Once the catheter system 100 is positioned in the target position, the lacerator 202 may be rotated so that the lacerator 202 is oriented towards the target IVS 1108, using radiographic markers, and using x-ray fluoroscopy along with adjunctive imaging such as ultrasound. When it is confirmed that the lacerator 202 is oriented properly, tension/traction on the lacerator 202 may be applied from the handle 102 of the catheter system 100 along the anchor guidewire to embed the lacerator 202 in the target myocardium/IVS 1108 (which may be referred to as “diving”). Electrosurgery may be applied to the lacerator 202 to assist embedding. In some examples, a 5% dextrose-saline solution or another nonionic liquid may be injected from the catheter system 100 along the lacerator 202 to displace blood and assure electrosurgery along the lacerator-myocardial contact point only. Some lacerator radial obliquity is expected at the initial incision point, because the lacerator 202 is guided only by manual operator torque-orientation of the catheter system. With traction-withdrawal during electrosurgery, the catheter system 100 will embed/dive more deeply, applying appropriate torque to achieve orthogonal radial orientation until the self-orientation/asymmetric hoop elements both appose the endocardium and assure orthogonal radial orientation. The lacerator depth may be adjusted as needed along the longitudinal laceration/scoring trajectory. Electrosurgery may continue to be applied for distal-to-proximal longitudinal laceration/scoring. The position/movement of the outer catheter shaft 104 may be monitored, if any, along the basal septal target, to protect the aortic valve apparatus from inadvertent injury. Further, the second set of hoop elements 118 may act to protect the aortic valve from inadvertent injury. During and/or after incision, the heart and the laceration may be visualized using cardiac imaging such as echocardiography.

Thus, the catheter system 100 shown in FIGS. 1-11 may be deployed to perform a myotomy on myocardium to relieve or prevent LVOT obstruction, for example. The catheter system 100 may include an incision catheter that comprises an articulated lacerator that, when deployed, extends at an oblique angle relative to the incision catheter and is configured to move along a longitudinal incision trajectory. The catheter system 100 may further include an anchor stabilization and orientation catheter system with at least one anchor element configured to be positioned in a heart to guide the incision catheter and orient the incision trajectory. Specifically, the catheter system 100 includes two anchor elements in the form of sets of endocameral stabilizing hoop elements (e.g., the first set of hoop elements 116 and the second set of hoop elements 118) with the incision catheter positioned intermediate the two sets of hoop elements (in a longitudinal direction). The lacerator of the incision catheter may be moved longitudinally between the two sets of endocameral stabilizing hoop elements along the incision trajectory from a distal position proximate the first set of hoop elements to a proximal position in a direction toward the second set of hoop elements. While the catheter system 100 has been described herein as being configured for performing SESAME procedures, it is to be appreciated that the catheter system 100 may be used for other cardiac procedures, such as delivery of lengthwise slices in non-septal locations, e.g., to address heart failure with preserved ejection fraction.

In some examples, the anchor and stabilization catheter system may include one or more transmural anchors to anchor and stabilize the incision catheter, rather than the two sets of endocameral hoop elements described above. FIGS. 12A and 12B show another myotomy catheter system 1200 (also referred to as a TAHINI catheter system) according to a second embodiment of the disclosure. Catheter system 1200 may include an outer catheter shaft 1202 that may be coupled at a proximal end to a handle (not shown in FIG. 12A), which may be integrated with outer catheter shaft 1202 or separate from but couplable to outer catheter shaft 1202. Similar to handle 102, the separate or integrated handle of the catheter system 1200 may include aspects to control the incision catheter (extend or retract the catheter/lacerator, including variably and interactively), and to electrify the electrosurgical traversal and laceration surface, and optionally to indicate the rotational position based radiographic attenuating markers and/or to inject non-ionic flush or angiographic contrast. The outer catheter shaft 1202 houses an incision catheter 1204 that includes a lacerator 1206. The incision catheter 1204 and lacerator 1206 may be similar to the incision catheter 110 and lacerator 202 described above, and thus description of the incision catheter 110 and lacerator 202 provided above may likewise apply to the incision catheter 1204 and lacerator 1206. It should be appreciated that different parts of the catheter system 1200 may have separate handles and may be removed and reintroduced separately. For example, the incision catheter 1204 (and lacerator 1206) may be removable and re-introducible coaxially through or along the outer catheter shaft 1202, each having separate handles and hemostatic valves as appropriate (e.g., a first handle may be present to control the incision catheter and lacerator and a second handle may be present to control the transmural anchors that are described below). In other examples, one handle may control the incision catheter and lacerator and the transmural anchors.

The outer catheter shaft 1202 forms a central guidewire lumen that ends proximal to the distal lacerator 1206, allowing the lacerator 1206 to engage myocardium distal to the transmural anchors (which will be described below), which may allow laceration of left ventricular wall structures that extend beyond the right ventricular cavity, lengthwise. The outer catheter shaft 1202 includes anchor ports (not visible in FIG. 12A) to accommodate a set of transmural anchors 1208 that may exit the outer catheter shaft 1202 at a side exit. The set of transmural anchors 1208 includes a first transmural guidewire 1208a that may exit the outer catheter shaft 1202 via a first anchor port and a second transmural guidewire 1208b that may exit the outer catheter shaft 1202 via a second anchor port. The set of transmural anchors 1208 may exit the outer catheter shaft 1202 on the same side of the catheter system 1200 as the lacerator 1206 (e.g., on the lacerator side). The set of transmural anchors 1208 may extend alongside (e.g., in parallel to) each other and at an angle relative to the outer catheter shaft 1202. When the lacerator 1206 is moved longitudinally during laceration, the lacerator 1206 may move along a trajectory that is parallel to the central longitudinal axis of the catheter system 1200 and that is oriented by the transmural anchors, e.g., the trajectory may extend between the first transmural guidewire 1208a and the second transmural guidewire 1208b (e.g., the trajectory, and in some examples the lacerator itself, may be positioned with the first transmural guidewire on a first side, such as a left side, and the second transmural guidewire on a second side, such as a right side, of the trajectory). The set of transmural anchors 1208 may be placed radially (orthogonal to the endocardial surface) into the right ventricular space (as will be described in more detail below).

FIG. 12C shows positioning of the catheter system 1200 in the heart 1102. To position the catheter system 1200 in the heart 1102, a guiding catheter (not shown) may be used to direct the set of transmural anchors 1208 across the LV wall and into the right ventricular space 1110, where the transmural anchors can be left loose, ensnared, or replaced with temporary screw-in anchors. Using the two anchored transmural anchors (e.g., the first transmural guidewire 1208a and the second transmural guidewire 1208b), the lacerator 1206 is self-oriented toward the target myocardium, which the lacerator engages and lacerates (e.g., with application of electricity to perform electrosurgery). One or more snare catheters (such as snare catheter 1210) may ensnare and apply counter-traction to the transmural anchors. The catheter system 1200 may have two side-by-side monorail or over-the-wire guidewire lumens to orient itself along the guidewire rails, as depicted in FIG. 16 and described below.

Thus, the set of transmural anchors 1208 may be positioned via a retrograde transaortic guiding catheter across the IVS 1108 and into the right ventricular space 1110. The IVS 1108 may be traversed by the set of transmural anchors 1208 using mechanical or electrosurgical traversal. The set of transmural anchors 1208 can be navigated into the right ventricular space, where each can be ensnared to allow countertraction of a guidewire rail for performing a SESAME procedure. In some examples, dedicated anchor devices can be implanted in the IVS 1108 at the anchor location, to allow traction on a thin (such as 0.014″) guidewire used as above. In some examples, the dedicated anchor devices may be helical temporary anchors that disperse traction forces to reduce myocardial injury. In some examples, the snaring catheter system is deployed to indicate the position of the right ventricular endocardial surface and thereby indicate an unacceptable laceration depth. As explained above with respect to FIG. 11, the IVS 1108 exhibits septal hypertrophy causing a narrow LVOT. The catheter system 1200 is positioned with its distal tip near the distal laceration/scoring target, before the lacerator is deployed. The set of transmural anchors 1208 act as dual radial anchor guidewire rails to correctly orient the lacerator 1206 by the two rails to be orthogonal to the intended laceration.

FIGS. 13A-13E show another myotomy catheter system 1300 (also referred to as a TAHINI catheter system) according to a third embodiment of the disclosure. As shown in FIGS. 13A and 13B, catheter system 1300 may include an outer catheter shaft 1308 that may be coupled at a proximal end to a handle (not shown in FIGS. 13A or 13B). Similar to handle 102, the handle of the catheter system 1300 may include aspects to control the incision catheter (extend or retract the catheter/lacerator, including variably and interactively), and to electrify the electrosurgical traversal and laceration surface, and optionally to indicate the rotational position based radiographic attenuating markers and/or to inject non-ionic flush or angiographic contrast. The outer catheter shaft 1308 houses an incision catheter 1310 that includes a lacerator 1312. The incision catheter 1310 and lacerator 1312 may be similar to the incision catheter 110 and lacerator 202 described above, and thus description of the incision catheter 110 and lacerator 202 provided above may likewise apply to the incision catheter 1310 and lacerator 1312.

The outer catheter shaft 1308 forms a central guidewire lumen. The outer catheter shaft 1308 includes anchor ports (not visible in FIG. 13A or 13B) to accommodate a set of transmural anchors comprised of a first transmural guidewire 1302 and a second transmural guidewire 1306 that each exit the outer catheter shaft 1308 at a side exit. The first transmural guidewire 1302 may exit the outer catheter shaft 1308 via a first anchor port positioned distally relative to a second anchor port via which the second transmural guidewire 1306 may exit the outer catheter shaft 1308. In this way, the first transmural guidewire 1302 may be longitudinally offset/spaced apart from the second transmural guidewire 1306, with the first transmural guidewire 1302 exiting the outer catheter shaft 1308 closer to the incision catheter 1310 than the second transmural guidewire 1306. The set of transmural anchors may exit the outer catheter shaft 1308 on the same side of the catheter system 1300 as the lacerator 1312 (e.g., on the lacerator side). The set of transmural anchors may extend alongside (e.g., in parallel to) each other and at an angle relative to the outer catheter shaft 1308. When the lacerator 1312 is moved longitudinally during laceration, the lacerator 1312 may move along a trajectory that is parallel to the central longitudinal axis of the catheter system 1300 and is oriented by the first transmural guidewire 1302 and the second transmural guidewire 1306 (e.g., the trajectory may be positioned with the first transmural guidewire on a first side, such as a left side, and the second transmural guidewire on a second side, such as a right side, of the trajectory). Additionally, along at least a portion of the incision trajectory, the lacerator may move longitudinally between the first transmural guidewire and the second transmural guidewire. The set of transmural anchors may be placed radially (orthogonal to the endocardial surface) into the right ventricular space (as will be described in more detail below).

FIGS. 13C-13E show positioning of the catheter system 1300 in the heart 1102. To position the catheter system 1300 in the heart 1102, a guiding catheter 1304 may be used to direct the first transmural guidewire 1302 across the LV wall and into the right ventricular space 1110 (as shown in FIG. 13C), where the transmural guidewire can be left loose, ensnared, or replaced with a temporary screw-in anchor. With the first transmural guidewire 1302 in place, the second transmural guidewire 1306 is placed, as shown in FIG. 13D. The second transmural guidewire 1306 may be positioned across the IVS 1108 and into the right ventricular space 1110, but closer to the aortic valve 1114 than the first transmural guidewire 1302, which may be positioned closer to the apex.

The outer catheter shaft 1308 and incision catheter 1310 are then positioned along the anchored guidewires. Using the two anchored transmural anchors (e.g., the first transmural guidewire 1302 and the second transmural guidewire 1306), the lacerator 1312 is oriented toward the target myocardium, which the lacerator engages and lacerates (e.g., with application of electricity to perform electrosurgery). One or more snare catheters (such as snare catheters 1314 and 1316 shown in FIG. 13E) may ensnare and apply counter-traction to the transmural anchors. The catheter system 1300 may have two monorail or over-the-wire guidewire lumens to orient itself along the guidewire rails, one proximal and one distal.

Thus, the set of transmural anchors of the catheter system 1300 may be positioned via a retrograde transaortic guiding catheter across the IVS 1108 and into the right ventricular space 1110. The IVS 1108 may be traversed by the set of transmural anchors using mechanical or electrosurgical traversal. The set of transmural anchors can be navigated into the right ventricular space, where each can be ensnared to allow countertraction of a guidewire rail for performing a SESAME procedure. In some examples, dedicated anchor devices can be implanted in the IVS 1108 at the anchor location, to allow traction on a thin (such as 0.014″) guidewire used as above. In some examples, the dedicated anchor devices may be helical temporary anchors that disperse traction forces to reduce myocardial injury. In some examples, the snaring catheter system is deployed to indicate the position of the right ventricular endocardial surface and thereby indicate an unacceptable laceration depth. As explained above with respect to FIG. 11, the IVS 1108 exhibits septal hypertrophy causing a narrow LVOT. The catheter system 1300 is positioned with its distal tip near the distal laceration/scoring target, before the lacerator is deployed. The set of transmural anchors act as dual radial anchor guidewire rails to correctly orient the lacerator 1312 by the two rails to be orthogonal to the intended laceration.

FIGS. 14 and 15 show a deployed lacerator 1206 (blue arrow) engaging myocardium upon traction (dotted green arrow) over a transmural guidewire (e.g., a guidewire of the set of transmural anchors 1208). While FIGS. 14 and 15 depict a single transmural guidewire, it is to be appreciated that dual guidewires could be used, as shown in FIGS. 12A-12C or FIGS. 13A-13E. Further, a similar engagement and traction may occur for the catheter system 100.

While the catheter system 1200 and the catheter system 1300 each include a set of mural anchors that includes two transmural guidewires, in some examples, a catheter system may only include one guidewire. In such examples, the catheter system may include two orienting guidewire ports to deploy guidewires or other metallic guidewires on opposing sides (e.g., spaced apart by 180 degrees or less, as depicted in FIG. 21B) to stabilize the incision catheter. Further, rather than utilize radial guidewire anchor(s), a catheter system may instead use a longitudinal guidewire. In such examples, the longitudinal guidewire may be navigated through the septal myocardium towards the apex until the guidewire exits the myocardium and re-enters the left ventricular space. The guidewire is then ensnared (e.g., by a snare device positioned distally on the catheter system), externalized, and used as a guidewire rail to engage the left ventricular endocardium with the incision catheter.

FIG. 16 schematically shows the positioning of an incision catheter (the black circles) relative to myocardium in a dual-guidewire embodiment. The dual-guidewire embodiment includes two guidewire ports to accommodate two guidewires and the incision catheter is positioned between the guidewire ports, with the two guidewire ports and the incision catheter positioned along the mycardium. FIG. 17 shows the incision catheter and deployed lacerator with oblique initial engagement of the lacerator relative to the myocardium.

Thus, the TAHINI catheter systems provided herein may provide for controlled or fixed-depth myotomy of myocardium to prevent or reduce instances of endomyocardial scoring that are too shallow or too deep. The catheter systems described herein include an incision catheter that may allow for angulated retro-engagement (like a plough-share) using a retractable articulated lacerator. Retraction allows for delivery of the lacerator into heart for deployment. Once extended and apposed to the endocardial surface and advanced/withdrawn, the angulated articulated lacerator advances deep into myocardium until an allowable maximum depth is reached. The articulated lacerator includes push-pull cables or a rod integrated into the catheter. The lacerator allows mechanical and electrosurgical laceration, and may be insulated to concentrate charge along the leading edge of the lacerator.

The catheter systems disclosed herein further include an anchor and stabilization catheter system that may include a double-stabilizer-element guiding catheter including two sets of endocameral stabilizing hoop elements that may be expanded to engage heart tissue and provide proper positioning/orientation of the incision catheter and rotational stabilization of the incision catheter and lacerator included therein. In other examples, the anchor and stabilization catheter system may include anchoring transmural guidewire(s) that may be positioned across the interventricular septum to provide proper positioning/orientation of the incision catheter and rotational stabilization of the incision catheter and lacerator. In examples where the catheter system includes anchoring transmural guidewire(s), the catheter system may include an anchor port(s) proximal to the distal end of the catheter system, which may allow the incision catheter to be positioned distal to the guidewire port(s) to address the limited distal longitudinal extent of the laceration due to the relation of the right and left ventricles along the septum. Further, in some examples, the catheter system may include balloon inflation/deflation ports or nitinol wing stabilizers, particularly in examples where a single guidewire anchor is utilized.

FIGS. 18A-18D show a second example myotomy catheter system 1800 (also referred to as a TAHINI catheter system) similar to the first embodiment of the disclosure. FIG. 18A shows a side view of the catheter system 1800, FIG. 18B shows a bottom view (e.g., of a lacerator side) of the catheter system 1800, FIG. 18C shows a distal end view of the catheter system 1800, and FIG. 18D shows a distal end view of the catheter system 1800 with the handle removed for clarity. FIGS. 18A-18D include a coordinate system 1899 to orient the views. Catheter system 1800 may be similar to catheter system 100 and thus may include a handle 1802, an outer catheter shaft 1804, a first inner tube 1806, a second inner tube 1808 (shown in FIG. 20), an incision catheter 1810 including a deployable lacerator 1803, a third inner tube 1811, and a fourth inner tube 1812 that may act as a guidewire lumen to accommodate a guidewire 1814. The description of the handle, outer catheter shaft, first inner tube, second inner tube, incision catheter, third inner tube, and fourth inner tube provided above with respect to FIGS. 1-11 likewise applies to the catheter system 1800.

The catheter system 1800 includes two sets of endocameral stabilizing hoop elements, similar to the catheter system 100, including a first set of hoop elements 1816 and a second set of hoop elements 1818. The first set of hoop elements 1816 may be similar to the first set of hoop elements 116 of catheter system 100, and thus the description of the first set of hoop elements 116 provided above with respect to FIGS. 1-11 likewise applies to the first set of hoop elements 1816. The second set of hoop elements 1818 may be positioned similarly to the second set of hoop elements 118 (e.g., proximal to the incision catheter 1810). The second set of hoop elements 1818 includes two hoop elements that are spaced apart longitudinally and that extend outward on opposite sides of the first inner tube 1806. The second set of hoop elements 1818 includes a first hoop element 1818a positioned on a distal end of the first inner tube 1806 and a second hoop element 1818b positioned proximal the first hoop element 1818a, such that the first hoop element 1818a is positioned closer to the incision catheter 1810 than the second hoop element 1818b. In the position shown in FIG. 18A, each of the first hoop element 1818a and the second hoop element 1818b extend outward from the first inner tube 1806 with a similar width, though the degree of extension of the hoop elements may be controlled by adjusting the position of the first inner tube 1806 and/or other elements of the catheter system 1800.

As shown in FIG. 18B, the second hoop element 1818b may extend outward from the first inner tube 1806 on the lacerator side of the catheter system 1800, while the first hoop element 1818a may extend outward from the first inner tube 1806 on an opposite (e.g., top) side of the catheter system 1800. The first hoop element 1818a may have a figure-eight shape, such that the first hoop element 1818a is comprised of two hoops that intersect at an apex of the first hoop element 1818a. The second hoop element 1818b may be comprised of two hoops that extend from a common location (e.g., the central longitudinal axis) at a slight angle (e.g., 5 degrees) relative to each other. When viewed from the distal end (as shown in FIGS. 18C and 18D), the two hoops of the second hoop element 1818b frame the lacerator 1803.

Thus, in the catheter system 1800, the LVOT/RCC hoop configuration is modified to have two hoop elements, one of which is longitudinally offset from the other so that it straddles the aortic valve without interfering with aortic valve function. The more proximal (aortic root) hoop element (the second hoop element 1818b) naturally orients in the right Sinus of Valsalva (also referred to as the right aortic cusp) and its diameter can independently be adjusted. The more distal hoop element (e.g,. the first hoop element 1818a) may be positioned “below” the aortic valve and provides counterpressure against the aortic root hoop element, as shown in FIG. 20 and described in more detail below.

FIGS. 19A-19D show a third example catheter system 1900 that is similar to the second example catheter system 1800, but with the second hoop element 1918b provided at a different angle relative to the lacerator 1803 than shown in FIGS. 18A-18D. FIG. 19A shows a side view of the catheter system 1900, FIG. 19B shows a top view of the catheter system 1900, FIG. 19C shows a distal end view of the catheter system 1900, and FIG. 19D shows a distal end view of the catheter system 1900 with the handle removed for clarity. FIGS. 19A-19D include the coordinate system 1899 to orient the views. In the example shown in FIGS. 19A-19D, the keying elements of the catheter system 1900 (e.g., keying slots and/or protrusions) are positioned so as to maintain the second hoop element 1918b at an angle (e.g., 30 degrees) relative to the lacerator 1803. For example, the second hoop element 1918b is positioned so that the two hoops of the second hoop element 1918b do not extend in parallel to the lacerator 1803 (as shown in FIGS. 18A-18D), such that the second hoop element 1918b is positioned on a side of the lacerator 1803 instead of framing the lacerator 1803. Further, FIG. 19B shows a top side (opposite the lacerator side), where the figure-eight shape of the first hoop element 1818a can be seen.

FIG. 20 shows the catheter system 1800 positioned in the heart 1102. As described above, the first set of hoop elements 1816 may engage the apex 1106 to provide rotational stability to the catheter system. Further, the first hoop element 1818a is positioned below the aortic valve 1114 while the second hoop element 1818b is oriented in the right Sinus of Valsalva/aortic cusp. The positioning of the first hoop element 1818a and the second hoop element 1818b imparts rotational stability to the catheter system 1800 and allows for proper orientation of the lacerator 1803 relative to the target myocardium. The lacerator may be deployed, electrified, and moved along its incision trajectory in a manner similar to the lacerator 202 of the catheter system 100 and described above with respect to FIG. 11. FIGS. 22A and 22B show the rotational orientation of the catheter system 1800 relative to a heart. Specifically, FIGS. 22A and 22B show a depiction of the catheter system 1800 overlaid on computed tomography (CT) images of a candidate for SESAME. FIG. 22A shows the RCC, with the endocameral stabilizing hoop elements in place. FIG. 22B shows a parallel short-axis slice of the LV showing the lacerator engaged into septum.

FIGS. 21A-21D show another myotomy catheter system 2100 (also referred to as a TAHINI catheter system) according to a fourth embodiment of the disclosure. FIG. 21A shows a side view of the catheter system 2100, FIG. 21B shows a top view of the catheter system 2100, FIG. 21C shows a distal end view of the catheter system 2100, and FIG. 21D shows a distal end view of the catheter system 2100 with the handle removed for clarity. FIGS. 21A-21D include the coordinate system 1899 to orient the views. Catheter system 2100 may be similar to catheter system 1200 and thus may include an outer catheter shaft 2102, an incision catheter 2104, a lacerator 2106, and a handle 2110 that are similar to the outer catheter shaft, incision catheter, lacerator, and handle of the catheter system 1200, the description of which provided above likewise applies to catheter system 2100. Catheter system 2100 further includes a set of deployable myocardial anchors 2108 comprising a first deployable anchors2108a and a second deployable anchors2108b that each extend outward on the lacerator side of the outer catheter shaft 2102, near the incision catheter 2104, from respective anchor ports. Each deployable anchors may extend at an angle relative to the lacerator 2106 (e.g., 45 degrees and negative 45 degrees) such that the lacerator 2106 is framed by the deployable anchors 2108. The deployable anchors 2108 may be comprised of elastic metallic material (e.g., steel or nitinol) that, once released, engage/penetrate the septal myocardium to provide a stabilization in terms of position and rotation.

Thus, the catheter systems described herein are lacerating catheters with a lacerator (e.g., blade) that can be delivered and deployed such that it is positioned tangential to heart muscle. The lacerator may have a controllable laceration depth from the endocardial surface and can be positioned/stabilized in the heart using mural anchors (that enter or traverse the wall of the heart), as shown in FIGS. 12A-13E and 21A-21D, for example, or using endocameral stabilizing hoops (that stabilize the catheter within a heart chamber), as shown in FIGS. 1-11 and 18A-19D, for example. In some examples, a hybrid design may be used where both mural anchors and endocameral stabilizing hoops are included on the same catheter system, for example with distal mural anchors and proximal endocameral stabilizing hoop elements.

The TAHINI catheter system described herein may have multiple potential uses. For example, the TAHINI catheter system described herein may be used for septal reduction for hypertrophic cardiomyopathy (HCM) to reduce LVOT obstruction causing exercise intolerance and causing mitral valve regurgitation through systolic anterior motion of the anterior mitral valve leaflet, as well as to provide enlargement of the LVOT to allow transcatheter mitral valve replacement/implantation (TMVR). The septal scoring described herein performed by the TAHINI catheter may be performed as preparation for TMVR or as bailout to treat unexpected LVOT obstruction after TMVR. Further, the TAHINI catheter system may be used for enlargement of the LVOT to prevent dynamic LVOT obstruction after transcatheter aortic valve implantation (TAVR). Additionally, the TAHINI catheter system may be used to perform left ventricular endomyocardial scoring to reduce left ventricular compliance causing elevated left ventricular filling pressures, manifest as symptomatic heart failure with preserved ejection fraction (HFpEF), and/or to perform scoring of right ventricular outflow tract (RVOT) in subvalvular pulmonic stenosis.

FIGS. 1-10, 12A, 12B, 13D, 13D, 18A-18D, 19A-19D, and 21A-21D show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

In another representation, a myotomy catheter system includes an incision catheter including an articulated lacerator that, when deployed, extends at an oblique angle relative to the incision catheter and is configured to move along a longitudinal incision trajectory, an inner coaxial two-tube system to expand and contract a first set of hoop elements, the inner coaxial two-tube system positioned distal to the incision catheter, and an outer coaxial two-tube system to expand and contract a second set of hoop elements, the outer coaxial two-tube system positioned proximal to the incision catheter.

The disclosure also provides support for a catheter-based heart incision apparatus, comprising: an anchor stabilization and orientation catheter system with at least one anchor element, and an incision catheter including a lacerator configured to move along an incision trajectory oriented by the at least one anchor element. In a first example of the apparatus, the apparatus further comprises: an outer catheter shaft, wherein the incision catheter extends outward from the outer catheter shaft, and wherein the at least one anchor element comprises two transmural anchors configured to extend outward from the outer catheter shaft alongside other and at an angle relative to the incision catheter, with the lacerator configured to move along the incision trajectory between the two transmural anchors. In a second example of the apparatus, optionally including the first example, the incision catheter is positioned distal relative to a position on the outer catheter shaft where the two transmural anchors exit the outer catheter shaft. In a third example of the apparatus, optionally including one or both of the first and second examples, the at least one anchor element comprises a first set of endocameral stabilizing hoop elements and a second set of endocameral stabilizing hoop elements, the first set of hoop elements positioned at a distal end of the anchor stabilization and orientation catheter system and the second set of hoop elements positioned at a proximal end of the anchor stabilization and orientation catheter system, with the lacerator configured to be positioned tangential to a target myocardium and move along the incision trajectory between the distal end and the proximal end. In a fourth example of the apparatus, optionally including one or more or each of the first through third examples, the first set of hoop elements is coupled to a first inner tube of the anchor stabilization and orientation catheter system and is configured to be extended or retracted based on movement of a second inner tube of the anchor stabilization and orientation catheter system, the first and second inner tubes accommodated within and extending outward from the incision catheter. In a fifth example of the apparatus, optionally including one or more or each of the first through fourth examples, the apparatus further comprises: a third inner tube and an outer catheter shaft, the incision catheter extending outward from the third inner tube, and wherein the second set of hoop elements is coupled to a fourth inner tube of the anchor stabilization and orientation catheter system and is configured to be extended or retracted based on movement of the outer catheter shaft, the third inner tube and the fourth inner tube accommodated within and extending outward from the outer catheter shaft. In a sixth example of the apparatus, optionally including one or more or each of the first through fifth examples, the at least one anchor element, when deployed, is configured to position the lacerator at an angle relative to septal myocardium of a patient. In a seventh example of the apparatus, optionally including one or more or each of the first through sixth examples, the lacerator includes a blade that, when deployed, is configured to extend at an oblique angle relative to a longitudinal axis of the incision catheter. In an eighth example of the apparatus, optionally including one or more or each of the first through seventh examples, the blade is configured to extend within a range of 0 to 60 degrees relative to the longitudinal axis. In a ninth example of the apparatus, optionally including one or more or each of the first through eighth examples, the apparatus further comprises: a handle including an actuating element coupled to the lacerator, the lacerator configured to extend or retract from the incision catheter and/or advance or retract along the incision trajectory based on actuation of the actuating element, and wherein the lacerator is comprised of electrically-conductive material and is configured to transmit ablative electrosurgical energy. In a tenth example of the apparatus, optionally including one or more or each of the first through ninth examples, the apparatus further comprises: one or more keying elements to maintain a rotational position of the incision catheter relative to the anchor stabilization and orientation catheter system.

The disclosure also provides support for a catheter-based heart incision apparatus, comprising: an incision catheter including an articulated lacerator that, when deployed, extends at an oblique angle relative to the incision catheter and is configured to move along a longitudinal incision trajectory, and an anchor stabilization and orientation catheter system with at least one anchor element configured to be positioned into a heart chamber and/or or across heart muscle to guide the incision catheter. In a first example of the apparatus, the at least one anchor element includes at least one transmural guidewire. In a second example of the apparatus, optionally including the first example, the apparatus further comprises: at least one guidewire lumen configured to accommodate the at least one transmural guidewire, wherein the at least one guidewire lumen ends proximal to the lacerator. In a third example of the apparatus, optionally including one or both of the first and second examples, the at least one anchor element includes at least one set of endocameral stabilizing hoop elements. In a fourth example of the apparatus, optionally including one or more or each of the first through third examples, the at least one set of endocameral stabilizing hoop elements includes a first set of hoop elements positioned distal to the lacerator and a second set of hoop elements positioned proximal to the lacerator. In a fifth example of the apparatus, optionally including one or more or each of the first through fourth examples, the apparatus further comprises: a guidewire lumen configured to accommodate a guidewire, the guidewire lumen terminating distal to the first set of hoop elements. In a sixth example of the apparatus, optionally including one or more or each of the first through fifth examples, the lacerator is comprised of electrically-conductive material and the lacerator includes an insulating material over a portion of the lacerator to form an exposed monopole of the lacerator that is configured to effect electrosurgical laceration. In a seventh example of the apparatus, optionally including one or more or each of the first through sixth examples, the apparatus further comprises: a feedback electrode configured to provide feedback regarding a radial depth of the lacerator in the heart muscle.

The disclosure also provides support for a method for performing a cardiac myotomy procedure with a catheter system, comprising: placing at least one anchor element in a heart, advancing an incision catheter of the catheter system into a left ventricle of the heart, deploying a lacerator of the incision catheter at an oblique angle relative to myocardium of the heart, providing ablative electrosurgery energy to the lacerator, and withdrawing the lacerator along a longitudinal incision trajectory to incise the myocardium. In a first example of the method, placing the at least one anchor element in the heart comprises expanding a first set of endocameral stabilizing hoop elements of the catheter system at an apex of the left ventricle and expanding a second set of endocameral stabilizing hoop elements of the catheter system at a left ventricular outflow tract and/or aortic root of the heart. In a second example of the method, optionally including the first example, placing the at least one anchor element comprises positioning at least one transmural guidewire through the left ventricle, into an interventricular septum of the heart, and into a right ventricle of the heart, and wherein advancing the incision catheter comprises advancing the incision catheter along the at least one transmural guidewire. In a third example of the method, optionally including one or both of the first and second examples, the lacerator is maintained at the oblique angle during the withdrawal of the lacerator.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A catheter-based heart incision apparatus, comprising:
an anchor stabilization and orientation catheter system with at least one anchor element; and
an incision catheter including a lacerator configured to move along an incision trajectory oriented by the at least one anchor element.

2. The apparatus of claim 1, further comprising an outer catheter shaft, wherein the incision catheter extends outward from the outer catheter shaft, and wherein the at least one anchor element comprises two transmural anchors configured to extend outward from the outer catheter shaft alongside other and at an angle relative to the incision catheter, with the lacerator configured to move along the incision trajectory between the two transmural anchors.

3. The apparatus of claim 2, wherein the incision catheter is positioned distal relative to a position on the outer catheter shaft where the two transmural anchors exit the outer catheter shaft.

4. The apparatus of claim 1, wherein the at least one anchor element comprises a first set of endocameral stabilizing hoop elements and a second set of endocameral stabilizing hoop elements, the first set of hoop elements positioned at a distal end of the anchor stabilization and orientation catheter system and the second set of hoop elements positioned at a proximal end of the anchor stabilization and orientation catheter system, with the lacerator configured to be positioned tangential to a target myocardium and move along the incision trajectory between the distal end and the proximal end.

5. The apparatus of claim 4, wherein the first set of hoop elements is coupled to a first inner tube of the anchor stabilization and orientation catheter system and is configured to be extended or retracted based on movement of a second inner tube of the anchor stabilization and orientation catheter system, the first and second inner tubes accommodated within and extending outward from the incision catheter.

6. The apparatus of claim 5, further comprising a third inner tube and an outer catheter shaft, the incision catheter extending outward from the third inner tube, and wherein the second set of hoop elements is coupled to a fourth inner tube of the anchor stabilization and orientation catheter system and is configured to be extended or retracted based on movement of the outer catheter shaft, the third inner tube and the fourth inner tube accommodated within and extending outward from the outer catheter shaft.

7. The apparatus of claim 1, wherein the at least one anchor element, when deployed, is configured to position the lacerator at an angle relative to septal myocardium of a patient.

8. The apparatus of claim 1, wherein the lacerator includes a blade that, when deployed, is configured to extend at an oblique angle relative to a longitudinal axis of the incision catheter.

9. The apparatus of claim 8, wherein the blade is configured to extend within a range of 0 to 60 degrees relative to the longitudinal axis.

10. The apparatus of claim 1, further comprising a handle including an actuating element coupled to the lacerator, the lacerator configured to extend or retract from the incision catheter and/or advance or retract along the incision trajectory based on actuation of the actuating element, and wherein the lacerator is comprised of electrically-conductive material and is configured to transmit ablative electrosurgical energy.

11. The apparatus of claim 1, further comprising one or more keying elements to maintain a rotational position of the incision catheter relative to the anchor stabilization and orientation catheter system.

12. A catheter-based heart incision apparatus, comprising:
an incision catheter including an articulated lacerator that, when deployed, extends at an oblique angle relative to the incision catheter and is configured to move along a longitudinal incision trajectory; and
an anchor stabilization and orientation catheter system with at least one anchor element configured to be positioned into a heart chamber and/or or across heart muscle to guide the incision catheter.

13. The apparatus of claim 12, wherein the at least one anchor element includes at least one transmural guidewire, the apparatus further comprising at least one guidewire lumen configured to accommodate the at least one transmural guidewire, wherein the at least one guidewire lumen ends proximal to the lacerator.

14. (canceled)

15. The apparatus of claim 12, wherein the at least one anchor element includes at least one set of endocameral stabilizing hoop elements.

16. The apparatus of claim 15, wherein the at least one set of endocameral stabilizing hoop elements includes a first set of hoop elements positioned distal to the lacerator and a second set of hoop elements positioned proximal to the lacerator.

17. The apparatus of claim 16, further comprising a guidewire lumen configured to accommodate a guidewire, the guidewire lumen terminating distal to the first set of hoop elements.

18. The apparatus of claim 12, wherein the lacerator is comprised of electrically-conductive material and the lacerator includes an insulating material over a portion of the lacerator to form an exposed monopole of the lacerator that is configured to effect electrosurgical laceration.

19. The apparatus of claim 12, further comprising a feedback electrode configured to provide feedback regarding a radial depth of the lacerator in the heart muscle.

20. A method for performing a cardiac myotomy procedure with a catheter system, comprising:
placing at least one anchor element in a heart;
advancing an incision catheter of the catheter system into a left ventricle of the heart;
deploying a lacerator of the incision catheter at an oblique angle relative to myocardium of the heart;
providing ablative electrosurgery energy to the lacerator; and
withdrawing the lacerator along a longitudinal incision trajectory to incise the myocardium.

21. The method of claim 20, wherein:
placing the at least one anchor element in the heart comprises expanding a first set of endocameral stabilizing hoop elements of the catheter system at an apex of the left ventricle and expanding a second set of endocameral stabilizing hoop elements of the catheter system at a left ventricular outflow tract and/or aortic root of the heart, or
placing the at least one anchor element comprises positioning at least one transmural guidewire through the left ventricle, into an interventricular septum of the heart, and into a right ventricle of the heart, and wherein advancing the incision catheter comprises advancing the incision catheter along the at least one transmural guidewire, or
the lacerator is maintained at the oblique angle during the withdrawal of the lacerator.
